SEQUENCE LISTING (2 sequences)

SEQ ID NO: 1 - DNA, 4579 bp, Homo sapiens
  source 1..4579: mol_type = other DNA
agacacgacattgcagcactggtgaccggatccagagcggagccaggctgcagtggtggggaggaccgggcaggtccagatgactttgaagggactccagaggaaggagagagtggaggaggagggctttgggttagggagagtgctttcgtttgttttaaatgggagaaactggagcatgttgccaagggcagagagccagcagagaggggtgaatggaagaaggagcgagaagggggttactgacgaagccttatcctggaggagagaaggatggactccagagcccagctttggggactggccttgaataaaaggagggccactctacctcatcctggagggagcacgaacctaaaggcagacccagaagagctttttacaaaactagagaaaattgggaagggctcctttggagaggtgttcaaaggcattgacaatcggactcagaaagtggttgccataaagatcattgatctggaagaagctgaagatgagatagaggacattcaacaagaaatcacagtgctgagtcagtgtgacagtccatatgtaaccaaatattatggatcctatctgaaggatacaaaattatggataataatggaatatcttggtggaggctccgcactagatctattagaacctggcccattagatgaaacccagatcgctactatattaagagaaatactgaaaggactcgattatctccattcggagaagaaaatccacagagacattaaagcggccaacgtcctgctgtctgagcatggcgaggtgaagctggcggactttggcgtggctggccagctgacagacacccagatcaaaaggaacaccttcgtgggcaccccattctggatggcacccgaggtcatcaaacagtcggcctatgactcgaaggcagacatctggtccctgggcataacagctattgaacttgcaagaggggaaccacctcattccgagctgcaccccatgaaagttttattcctcattccaaagaacaacccaccgacgttggaaggaaactacagtaaacccctcaaggagtttgtggaggcctgtttgaataaggagccgagctttagacccactgctaaggagttattgaagcacaagtttatactacgcaatgcaaagaaaacttcctacttgaccgagctcatcgacaggtacaagagatggaaggccgagcagagccatgacgactcgagctccgaggattccgacgcggaaacagatggccaagcctcggggggcagtgattctggggactggatcttcacaatccgagaaaaagatcccaagaatctcgagaatggagctcttcagccatcggacttggacagaaataagatgaaagacatcccaaagaggcctttctctcagtgtttatctacaattatttctcctctgtttgcagagttgaaggagaagagccaggcgtgcggagggaacttggggtccattgaagagctgcgaggggccatctacctagcggaggaggcgtgccctggcatctccgacaccatggtggcccagctcgtgcagcggctccagagatactctctaagtggtggaggaacttcatcccactgaaattcctttggcatttggggttttgtttttccttttttccttcttcatcctcctccttttttaaaagtcaacgagagccttcgctgactccaccgaagaggtgcgccactgggagccaccccagcgccaggcgcccgtccagggacacacacagtcttcactgtgctgcagccagatgaagtctctcagatgggtggggagggtcagctccttccagcgatcattttattttattttattacttttgtttttaattttaaccatagtgcacatattccaggaaagtgtctttaaaaacaaaaacaaaccctgaaatgtatatttgggattatgataaggcaactaaagacatgaaacctcaggtatcctgctttaagttgataactccctctgggagctggagaatcgctctggtggatgggtgtacagatttgtatataatgtcatttttacggaaaccctttcggcgtgcataaggaatcactgtgtacaaactggccaagtgcttctgtagataacgtcagtggagtaaatattcgacaggccataacttgagtctattgccttgcctttattacatgtacattttgaattctgtgaccagtgatttgggttttattttgtatttgcagggtttgtcattaataattaatgcccctctcttacagaacactcctatttgtacctcaacaaatgcaaattttccccgtttgccctacgccccttttggtacacctagaggttgatttcctttttcatcgatggtactatttcttagtgttttaaattggaacatatcttgcctcatgaagctttaaattataattttcagtttctccccatgaagcgctctcgtctgacatttgtttggaatcgtgccactgctggtctgcgccagatgtaccgtcctttccaatacgattttctgttgcaccttgtagtggattctgcatatcatctttcccacctaaaaatgtctgaatgcttacacaaataaattttataacacgcttattttgcatactccttgaaatgtgactcttcagaggacagggcacctgctgtgtatgtgtggccgtgcgtgtgtactcgtggctgtgtgtgtgtgatgagacactttggaagactccagggagaagtccccaggcctggagctgccgagtgcccaggtcagcgccctggactgcttgcgcacttgctcaccgagatgatgcagttggaggttgctgatctgtgcgattgctgtagcggttgccggggaccttaagagttattttgcttctctggaaggggcctatgcttgctaggcaggcagccagtgtgtctgtttttcttggtttgctgtgggaccttgcttggcgagggggaaaatctctgggtttctggagtgggagggttcgtgcagcagctgttgactggtacatgaagcattcttttatgtttgttgaagctgatgattgacatctcccgtgggtgtgccagttcttgtggagttaagacaggatttttggaagcaaggaagttagtgggtgagcttggggatgtagctcagctatctgctggtctagtggcctctaagctatagggaggggacagagccctgagctacagatgcttgagtgggttattgtgtcggtttgctagtgcagtctggtttttaagctctaaaattgaggtattttattagaagtggatttgggttgaactcttaatttgtataagggatatattttggttggggaaatagaactgagttgctaattcttattgtactcattactccatacaagaatgttatgttgaataataaaattggagaagatttcattttgtgtttccagggagtattctgtgtggggaactgtttccttacgtgaggccggcggcataagtcaaagatgagttttgtccttgcgaatcacacagattgagtctgtgttccccagggtgtgccgttacctgatttttaagtgagccagggcggacagcagcttttctgatttacagagttcttcagatttacaaatggacaatgacatcacagtttttagcactgaagccagtctcatgctagtaacagtgggtgagccgctcgagggactgggttctaatgaatactggtatgaacggggagtctctgcagtcgccagacaaatcatactcagccccttcccccgtagagcaacaagtggttcttttagagttgactggcagcatttcctgtcgggggaggtggggtttgatggagttagaaagctcgcctctgtgtacattctctcctgggctgttactttctgtagacgcacaaaatcagccccaatgtttttaagggcatcttagccaaggaagctggcttttgtgtcgccacttccaggcctgcattaagagagagcccaggcaccagggctaccactggaacctgcctcagcgtcaactgctgctggtctgtagccaggcccagcctttgagacgggtttactgtcaccagtagcctctcagtgccagccctgagctgctcctggctcagctgcccagagcctgcagcctggggaggtactcagcctctgggagacgagggccgtggactgggtggctggtagctcctgcgtttttgagctgtgtcctggctggctgctgccaatgaggtggacaccagtgtggtttggggtgcactggccacttcttgctgggttctgattttcttggaagtgcatctgccttccttatccaatagttttatccctgcattgctcttgtgaagtggctggtttggttctgtatgtagcattttgtacctttcctctggcaaaacactgtcagtttataaacattttttatatttccctcctttaaaaacagcttgtgtatttctgctataaaatgtgtcagcaaaggcagagtgacctaatagggcatgttcttaagcacagggactgtatcatgcaggggccaataaagctcaagaaaacgagta

SEQ ID NO: 2 - protein, 443 aa, Homo sapiens
MDSRAQLWGLALNKRRATLPHPGGSTNLKADPEELFTKLEKIGKGSFGEVFKGIDNRTQKVVAIKIIDLEEAEDEIEDIQQEITVLSQCDSPYVTKYYGSYLKDTKLWIIMEYLGGGSALDLLEPGPLDETQIATILREILKGLDYLHSEKKIHRDIKAANVLLSEHGEVKLADFGVAGQLTDTQIKRNTFVGTPFWMAPEVIKQSAYDSKADIWSLGITAIELARGEPPHSELHPMKVLFLIPKNNPPTLEGNYSKPLKEFVEACLNKEPSFRPTAKELLKHKFILRNAKKTSYLTELIDRYKRWKAEQSHDDSSSEDSDAETDGQASGGSDSGDWIFTIREKDPKNLENGALQPSDLDRNKMKDIPKRPFSQCLSTIISPLFAELKEKSQACGGNLGSIEELRGAIYLAEEACPGISDTMVAQLVQRLQRYSLSGGGTSSH

(19) United States
(12) Patent Application Publication
(10) Pub. No.: US 2025/0189515 A1
(43) Pub. Date: Jun. 12, 2025

(54) METHODS FOR IDENTIFYING THERAPEUTIC AGENTS

(71) Applicant: ALEXERA AB, Göteborg (SE)

(72) Inventor: Margit MAHLAPUU, Göteborg (SE)

(21) Appl. No.: 19/063,933

(22) Filed: Feb. 26, 2025

Related U.S. Application Data
(63) Continuation of application No. 17/129,311, filed on Dec. 21, 2020.
Application No. 17/129,311 is a division of application No. 16/634,996, filed on Jan. 29, 2020, now abandoned.

(30) Foreign Application Priority Data
Sep. 13, 2017 (SE) 1730249-8

(51) Int. Cl. G01N 33/50; C12N 15/113; G01N 33/92

(52) U.S. Cl. CPC G01N 33/5038; C12N 15/113; G01N 33/92; C12N 2310/14; G01N 2500/04

(57) ABSTRACT

The present invention relates to methods for identifying compounds that can have an effect on lipid metabolism, and thereby have a high relevance for several human diseases including but not restricted to obesity, type 2 diabetes (T2D), non-alcoholic fatty liver disease (NAFLD), non-alcoholic steatohepatitis (NASH), fatty pancreas disease, and fatty kidney disease. More specifically, the present invention relates to methods for identifying modulators of the expression or the activity of the human kinase Mammalian Sterile20-like 3 (MST3) and use of such modulators in the treatment of metabolic disease.

### CROSS-REFERENCE TO RELATED APPLICATIONS

This application is a continuation of U.S. application Ser. No. 17/129,311, filed Dec. 21, 2020, which is a divisional of U.S. application Ser. No. 16/634,996, filed Jan. 29, 2020, which is a national stage application of International Appl. PCT/SE2018/050918, filed Sep. 12, 2018, and which claims priority of Swedish Patent Appl. No. 1730249-8, filed Sep. 13, 2017, all of which are hereby incorporated by reference.

### FIELD OF THE INVENTION

The present invention relates to methods for identifying compounds that can have an effect on lipid metabolism, and thereby have a high relevance for several human diseases including but not restricted to obesity, type 2 diabetes (T2D), non-alcoholic fatty liver disease (NAFLD), non-alcoholic steatohepatitis (NASH), fatty pancreas disease, and fatty kidney disease. More specifically, the present invention relates to methods for identifying modulators of the expression or the activity of the human kinase Mammalian Sterile20-like 3 (MST3).

### BACKGROUND TO THE INVENTION

The human kinome features a large branch of the so-called “STE” kinases, named after the yeast Sterile20 kinase. The STE superfamily includes several subfamilies, one of which is named the “Mammalian Sterile20-like” (MST) family (Creasy et al. 1996). There are five MST kinases in mammals, MST1 (also called STK4), MST2 (also called STK3), MST3 (also called STK24), MST4, and YSK1 (also called STK25 or SOK1). The five mammalian MST kinases can be broadly divided into three subgroups: MST1/2, MST3/4, and YSK1 depending on their respective regulation, substrates, and function (Thompson and Sahai, 2015).

MST1 and MST2 have been identified to be involved growth control, proliferation, and regulation of migration. MST3 and MST4 have been identified to influence cell migration, cell polarity, and apoptosis.

YSK1 has recently been shown to be a regulator of lipid and glucose metabolism (Nerstedt et al. 2012).

### Role of MST3 in Regulation of Cell Polarity and Migration

MST3 has been shown to regulate actin dynamics in many contexts. In the developing nervous system, MST3 is required for dendritic spine maintenance and limits filopodia formation (Ultanir et al. 2014). MST3 also limits actin-dependent protrusions in other cell types. This has been suggested to result in increased migration on 2D surfaces when it is depleted, but lead to defects in squeezing through gaps in 3D matrices (Lu et al. 2006; Madsen et al. 2015).

Mammalian cell culture studies also implicate MST3 in regulation of cell polarity. MST3 can localize to the Golgi apparatus possibly through interaction with Striatin proteins (Lu et al. 2006). Interaction with CCM3 or Mo25 can trigger the translocation of MST3 away from the Golgi apparatus to the plasma membrane.

### Role of MST3 in Regulation of Apoptosis

MST3 can be cleaved by caspases (Lee et al. 1998; Lee et al. 2001). The cleavage occurs at amino acid 313 and separates the N-terminal kinase domain from the C-terminal regulatory sequences. This results in nuclear accumulation of the active kinase domain, which can promote apoptosis (Huang et al. 2002; Lee et al. 2004).

### Role of MST3 in Disease

Consistent with its role in cell migration, MST3 has been implicated in cancer. It has been suggested that CCM3 promotes the activity of MST3 at the cell cortex, where it coordinates the phosphorylation of ERM proteins and MLC, enabling cancer cells to squeeze through small gaps (Madsen et al. 2015; Tozluoglu et al. 2015).

Proteomic work in mammalian cells has identified MST3 as a component of a large PP2A complex, termed the STRIPAK complex (Glatter et al. 2009; Kean et al. 2011). Recently, cancer genome sequencing has implicated the STRIPAK complex in cancer. FAM40B is mutated with a high frequency, and the number and type of mutations suggest that it has an oncogenic function (Davoli et al. 2013). Analysis of truncation mutants of FAM40B found in tumors reveals that they are not able to bind to the catalytic subunits of PP2A and may be defective in negatively regulating MST3 (Madsen et al. 2015).

Defective regulation of MST3 is also implicated in the pathology of endothelial malformations (Stockton et al. 2010; Zheng et al. 2010).

### Role of MST3 in Lipid Metabolism

The prior art fails to identify a role for MST3 in lipid metabolism.

### BRIEF SUMMARY OF THE INVENTION

A method for treating or preventing a metabolic disease in a subject is disclosed. The method comprises administering a pharmaceutically effective amount of an oligonucleotide or polynucleotide 8-80 bases in length. The oligonucleotide or polynucleotide is hybridizable to the nucleic acid sequence of SEQ ID NO:1, or a nucleic acid sequence complementary to SEQ ID NO:1, thereby suppressing intracellular accumulation of cholesteryl ester and treating the subject having a metabolic disease. The metabolic disease is selected from the group consisting of non-alcoholic fatty liver disease (NAFLD), non-alcoholic steatohepatitis (NASH), fatty pancreas disease, and fatty kidney disease.

In some embodiments, the metabolic disease is non-alcoholic fatty liver disease (NAFLD).

In some embodiments, the metabolic disease is non-alcoholic steatohepatitis (NASH).

In some embodiments, the oligonucleotide or polynucleotide is selected from the group consisting of siRNA, shRNA, and antisense RNA or DNA.

In some embodiments, the oligonucleotide or polynucleotide is antisense RNA or DNA.

In some embodiments, the antisense oligonucleotide or polynucleotide is 8-30 bases in length.

In some embodiments, the subject is human.

In some embodiments, a nucleotide in the oligonucleotide or polynucleotide comprises an RNA residue, a DNA residue, or a nucleotide analogue.

In some embodiments, the oligonucleotide or polynucleotide is selected from the group consisting of a peptide nucleic acid (PNA), a phosphorodiamidate morpholino oligomer (PMO), and a phosphorothioate oligonucleotide (PS-ODN).

### BRIEF DESCRIPTION OF THE DRAWINGS

FIGS. 1-4 show that depletion of MST3 in human hepatocytes suppresses lipid accumulation through increased β-oxidation and triacylglycerol (TAG) secretion combined with suppressed lipid uptake and synthesis. Immortalised human hepatocytes (IHHs) were transfected with MST3 or non-targeting control (NTC) small interfering RNA (siRNA); the assessments were performed after 48 h incubation with oleic acid (OA) and under basal conditions, as follows.

FIG. 1 shows (A) Representative Western blot with anti-MST3 antibodies, in which actin was used as a loading control; and (B) Oil Red O staining for neutral lipids, in which representative cell images are shown (scale bars, 100 μm).

FIG. 2 shows (A) Lipidomics analysis, [symbol] NTC siRNA Basal, [symbol] MST3 siRNA Basal, □ NTC siRNA+OA, ▪ MST3 siRNA+OA; and (B) Oxidation of radio-labelled palmitate.

FIG. 3 shows (A) Secretion of [³H]TAG into the media, -□- NTC siRNA, -▪- MST3

siRNA; and (B) Uptake of OA, -Δ- NTC siRNA Basal, -▴- MST3 siRNA Basal, -□- NTC siRNA+OA, -▪- MST3 siRNA+OA.

FIG. 4 shows (A) TAG synthesis from [¹⁴C]-labelled OA, and (B) TAG synthesis from [¹⁴C]-labelled glucose.

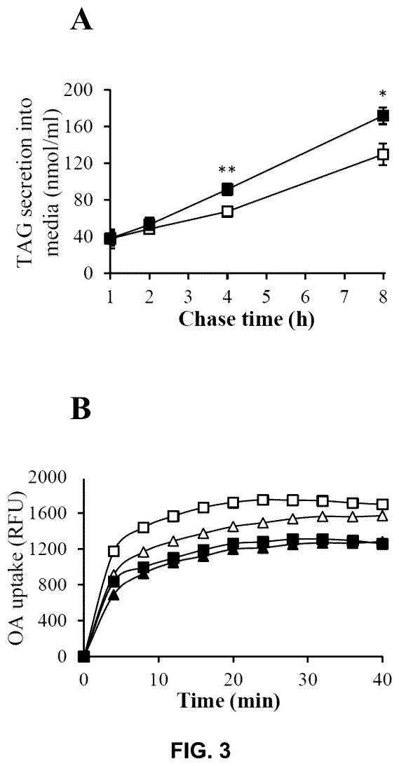

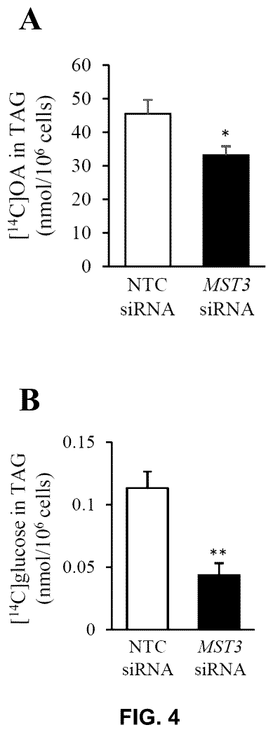

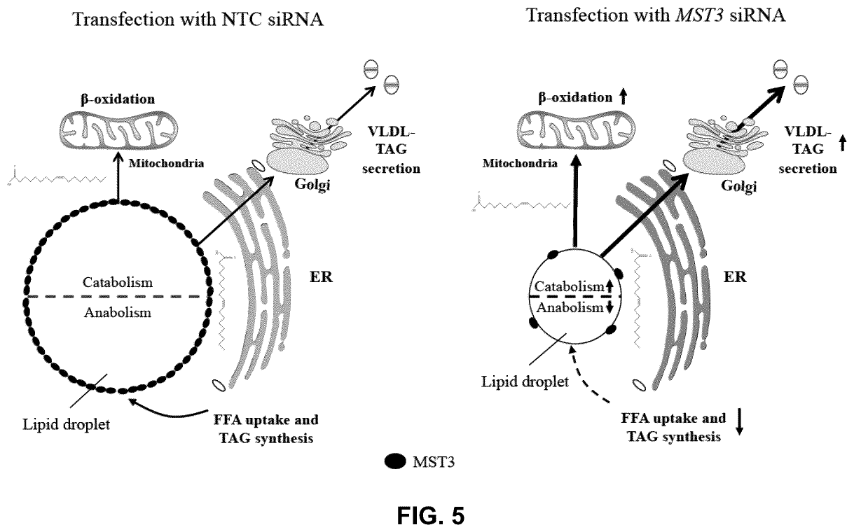

For FIG. 2, (A)-(B), FIG. 3, (A), and FIG. 4, (A)-(B), results are means±SEM from 8-10 cell culture dish wells. For FIG. 2, (B), FIG. 3, (A), and FIG. 4, (A)-(B), the assessments were performed only after 48 h incubation with OA.

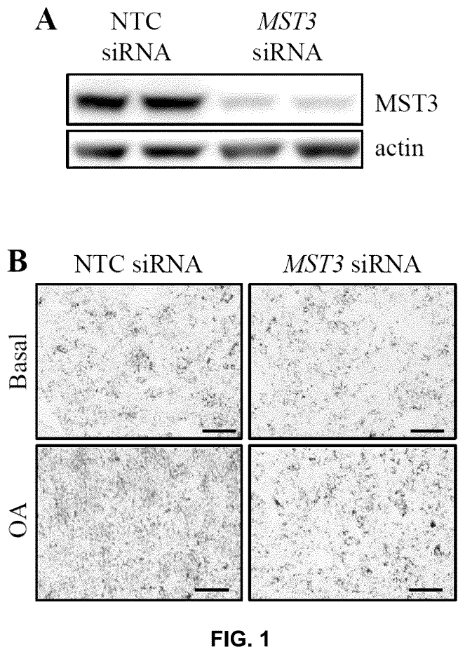

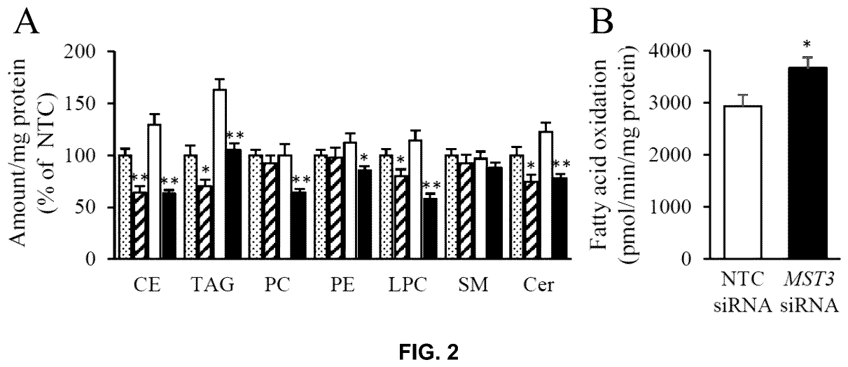

For FIGS. 1-4, abbreviations are as follows. CE, cholesteryl ester; Cer, ceramide; LPC, lysophosphatidylcholine; NEFA, non-esterified fatty acid; NTC, non-targeting control; OA, oleic acid; PC, phosphatidylcholine; PE, phosphatidyl-ethanolamine; RFU, relative fluorescence units; SM, sphingomyelin; TAG, triacylglycerol. *p<0.05; **p<0.01 (in FIG. 2, (A), only statistical significances comparing MST3 siRNA versus NTC siRNA in each test condition is shown).

FIG. 5 shows a schematic model for MST3 function in regulating lipid accumulation in human hepatocytes. Depletion of MST3 by siRNA enhances lipid droplet catabolism through increased β-oxidation and VLDL-TAG secretion, and represses lipid droplet anabolism through suppressed free fatty acid (FFA) uptake and TAG synthesis, compared with cells transfected with NTC siRNA. Consequently, MST3 deletion leads to lower intracellular TAG content and reduced lipid droplet size. ER, endoplasmic reticulum; FFA, free fatty acid; LD, lipid droplet, NTC, non-targeting control; TAG, triacylglycerol; VLDL, very-low-density lipoprotein.

### DETAILED DESCRIPTION

The present inventor has identified a role for MST3 in mammalian lipid metabolism. More specifically, the present inventor has recognized that MST3 has a role in regulation of lipid partitioning in mammalian cell system, and that MST3 controls the dynamic metabolic balance of lipid utilization versus lipid storage in peripheral tissues prone to lipotoxicity and is thereby expected to regulate insulin sensitivity, which has a high relevance for several human diseases including but not restricted to obesity, type 2 diabetes (T2D), non-alcoholic fatty liver disease (NAFLD), non-alcoholic steatohepatitis (NASH), fatty pancreas disease, and fatty kidney disease.

Accordingly, in one aspect the present invention provides methods for identifying modulators of mammalian lipid metabolism.

The methods comprise the use of mammalian MST3, preferably human MST3.

The methods can comprise determination of MST3 activity and/or MST3 expression.

MST3 activity and/or MST3 expression can be determined in mammalian cells expressing MST3. The mammalian cells can be cells with constitutive MST3 expression or cells overexpressing recombinant MST3.

The mammalian cells can be human or rodent cells.

In one embodiment the method according to the invention comprises a method for identifying an agent capable of modulating the activity and/or expression of MST3, which method comprises: (i) contacting a candidate compound modulator with a host-cell which expresses a polynucleotide sequence encoding a MST3 polypeptide; and (ii) determining an effect of the candidate compound modulator on the activity and/or expression of MST3 thereby identifying the compound as a MST3 modulator, and thereby identifying the compound as a potential modulator of mammalian lipid metabolism.

MST3 activity can be determined by measurement of lipid deposition, lipid catabolism e.g. fatty acid oxidation and/or lipid secretion, and/or lipid anabolism, e.g. lipid uptake and/or lipid synthesis. Since lipid deposition regulates insulin sensitivity, also the insulin sensitivity parameters such as insulin-stimulated glucose uptake (ISGU) can be used as a measure of MST3 activity.

MST3 expression can be determined using techniques such as quantitative real-time PCR (qRT-PCR), Western blot, or proximity ligation assay (PLA).

In another embodiment the method according to the invention comprises a method for identifying an agent capable of modulating the activity of MST3, which method comprises: (i) contacting a candidate compound modulator with a MST3 polypeptide; and (ii) determining an effect of the candidate compound modulator on the activity of MST3 to thereby identify the compound as a MST3 modulator, and thereby identifying the compound as a potential modulator of mammalian lipid metabolism.

MST3 activity can be determined using an MST3 polypeptide, preferably a recombinantly produced and purified MST3 polypeptide, and an MST3 peptide substrate, and determining the level of phosphorylation of the peptide substrate as a measure of MST3 activity.

In yet another embodiment the method according to the invention comprises a method for identifying an agent capable of modulating the activity of MST3, which method comprises: (i) contacting a candidate compound modulator with an MST3 polypeptide; and (ii) determining the binding of the candidate compound modulator to MST3 to thereby identify the compound as an MST3 binder, and thereby identifying the compound as a potential modulator of mammalian lipid metabolism.

MST3 binding can be determined using an MST3 polypeptide, preferably a recombinantly produced and purified MST3 polypeptide, and candidate compound, and determining the effect of the compound on the melting temperature of MST3 as described by Olesen et al. 2016.

The MST3 polypeptide can be a human MST3. The amino acid sequence of human MST3 can e.g. be found in UniProtKB database accession nr—Q9Y6E0 (STK24_HUMAN) and in the NCBI GenBank database accession nr NP_003567, here identified as SEQ ID NO:2.

The MST3 polypeptide according to the invention can comprise the amino acid sequence according to SEQ ID NO:2 or a variant of thereof having at least 80% sequence identity to the amino acid sequence according to SEQ ID NO:2 or a functionally active fragment thereof.

Functionally active fragments of MST3 are defined as MST3 polypeptides retaining at least 90% MST3 kinase activity, such as least 80%, 70%, 60%, or 50% MST3 kinase activity, as compared to the MST3 kinase activity of the MST3 polypeptide having the amino acid sequence according to SEQ ID NO:2.

Methods for screening for modulators of MST3 can be based on the use of a recombinantly produced and purified MST3 polypeptide and a MST3 peptide substrate. Enzymatic assays, such as TR-FRET or ADP hunter assay, can be adapted for MST3 assays, and used to determine the level of phosphorylation of the peptide substrate.

Selectivity of identified modulators can be assessed against a panel of kinases using standard radiometric filter plate assay. Surface plasmon resonance (SPR) can be used to assess binding of candidate compounds to MST3.

Candidate compounds which may be tested in the methods according to the invention include simple organic molecules, commonly known as “small molecules”, for example those having a molecular weight of less than 2000 Daltons. The methods may also be used to screen compound libraries such as peptide libraries, including synthetic peptide libraries and peptide phage libraries.

Once a modulator, i.e. an inhibitor or stimulator, of MST3 activity is identified then medicinal chemistry techniques can be applied to further refine its properties, for example to enhance efficacy and/or reduce side effects.

Other suitable candidate molecules include oligonucleotides and polynucleotides, such as dsRNA, siRNA, shRNA, miRNA and anti-sense RNA or DNA, and any other molecules which potentially can modulate the activity and/or expression of MST3.

The cDNA sequence encoding human MST can be e.g. found in the NCBI GenBank database accession nr NM_003576, here identified as SEQ ID NO:1.

Preferred oligonucleotides and polynucleotides consist of 8-80 bases in length, comprising a sequence hybridisable to the nucleic acid sequence SEQ ID NO:1.

It will be appreciated that there are many procedures known in the art which may be employed to perform the present invention. Examples of suitable procedures which may be used to identify a MST3 modulator include rapid filtration of equilibrium binding mixtures, enzyme linked immunosorbent assays (ELISA), radioimmunoassays (RIA) and fluorescence resonance energy transfer assays (FRET), scintillation proximity assay (SPA), electrophoretic mobility shift assay (EMSA), chromatin immunoprecipitation analysis (ChIP), surface plasmon resonance (SPR), qRT-PCR, Western blot, and PLA assays.

In a second aspect the present invention provides compounds, pharmaceutical composition, and methods for the treatment of metabolic diseases.

In one embodiment the invention provides oligonucleotides and polynucleotides consisting of 8-80 bases in length, comprising a sequence hybridisable to the nucleic acid sequence SEQ ID NO: 1 or the complementary sequences thereto for use in the treatment of metabolic diseases.

In another embodiment the invention provides a pharmaceutical composition comprising an oligonucleotide or a polynucleotide consisting of 8-80 bases in length, comprising a sequence hybridisable to the nucleic acid sequence SEQ ID NO:1 or the complementary sequences thereto for use in the treatment of metabolic diseases.

In another embodiment the invention provides oligonucleotides and polynucleotides consisting of 8-80 bases in length, comprising a sequence hybridisable to the nucleic acid sequence SEQ ID NO: 1 or the complementary sequences thereto for use in the manufacture of a medicament for the treatment of metabolic diseases.

In yet another embodiment the invention provides methods for treatment of metabolic diseases comprising administering a pharmaceutical effective amount of an oligonucleotide or a polynucleotide consisting of 8-80 bases in length, comprising a sequence hybridisable to the nucleic acid sequence SEQ ID NO:1 or the complementary sequences thereto to a subject in need of such treatment.

The metabolic disease can be selected from, but not limited to, obesity, type 2 diabetes (T2D), non-alcoholic fatty liver disease (NAFLD), non-alcoholic steatohepatitis (NASH), fatty pancreas disease, and fatty kidney disease.

Preferred oligonucleotides and polynucleotides are dsRNA, siRNA, shRNA, miRNA and anti-sense RNA or DNA.

The oligonucleotides and polynucleotides can be 8-80 bases in length, preferable 8-70, 8-60, 8-50, 8-40, or 8-30 bases in length.

A nucleotide in the antisense oligonucleotide or polynucleotide may be an RNA residue, a DNA residue, or a nucleotide analogue.

Antisense oligonucleotides and polynucleotides may be selected from the group consisting of a peptide nucleic acid (PNA), a phosphorodimidate mopholino oligomer (PMO), and a phosphorothioate oligonucleotide (PS-ODN).

### EXAMPLES

### Materials and Methods

Immortalised human hepatocytes (IHHs; liver cell line of human origin) were maintained in Complete William's E medium (Gibco, Paisley, UK) supplemented with bovine insulin (20 U/1; Sigma-Aldrich, St Louis, MO, USA) and dexamethasone (50 nmol/l; Sigma-Aldrich). Culture medium was also supplemented with 10% (vol./vol.) fetal bovine serum (FBS), L-glutamine (2 mmol/l), and 1% (vol./vol.) penicillin/streptomycin (Gibco). After transfections, cells were incubated with oleic acid (OA) for 48 h, which is known to efficiently induce steatosis in vitro (Amrutkar et al. 2016).

### MST3 Depletion

MST3 was depleted by small interfering RNA (siRNA) technique using Lipofectamine RNAiMax (Invitrogen, San Diego, CA, USA), according to manufacturer's instructions. The efficient target depletion was confirmed by Western blot using anti-MST3 antibodies.

### Measurement of Lipid Storage

To measure lipid storage, transfected cells were stained with Oil Red O for neutral lipids as previously described (Amrutkar et al. 2016). In addition, for lipidomic analysis, lipids were extracted using the Folch method (Folch et al. 1957) and quantified using ultraperformance liquid chromatography/mass spectrometry and direct-infusion mass spectrometry (Stahlman et al. 2013).

### Measurement of Fatty Acid Oxidation

To measure β-oxidation, transfected cells were incubated in the presence of [9,10-³H(N)]-palmitic acid, and [³H]-labelled water was measured as the product of free fatty acid oxidation as previously described (Nerstedt et al. 2012).

### Measurement of Triacylglycerol Secretion

To measure triacylglycerol (TAG) secretion, transfected cells were incubated with pulse media [Complete William's E containing 18,500 Bq/ml [³H]OA (PerkinElmer, Waltham, MA, USA), 360 μmol/l OA (Sigma-Aldrich), and 1% vol./vol. fatty acid-free BSA] for 8 h, followed by incubation with chase media (Complete William's E supplemented with 30% vol./vol. fatty acid-free BSA) for up to 8 h. Media was collected for lipid extraction, followed by lipid separation by thin-layer chromatography on silica gel plates. Radiolabelled TAG was detected by iodine vapor and quantified by a scintillation counter.

### Measurement of Oleic Acid Uptake

The oleic acid (OA) uptake was measured using the Quencher-Based Technology (QBT) Fatty Acid Uptake Assay Kit (Molecular Devices, San Jose, CA, USA), according to manufacturer's instructions.

Incorporation of Oleic Acid and Glucose into Triacylglycerol

The incorporation of [¹⁴C] OA and [¹⁴C] glucose into TAG was measured as previously described (Amrutkar et al. 2016).

Liquid Chromatography Mass Spectrometry (LC-MS) Analysis in Lipid Droplets from Mouse Liver

Male mice of C57BL6/J genetic background were fed a pelleted high-fat diet (45% kilocalories from fat; D12451; Research Diets, New Brunswick, NJ, USA) for 16-18 weeks. The mice were killed and lipid droplets (LDs) were isolated from freshly excised livers using the method of Zhang et al (Zhang et al. 2011). LC-MS analysis was performed as previously described (Chursa et al. 2017) with the following modifications. The proteins from LD extract were precipitated using ProteoExtract Kit (Millipore, Burlington, MA, USA) and dissolved in buffer [50 mmol/l triethylammonium bicarbonate (TEAB), 4% SDS] prior to protein concentration determination. Total protein TMT 10-plex sets were fractionated using Pierce High pH Reversed-Phase Peptide Fractionation Kit to 8 fractions prior to LC-MS analysis.

### Results

MST3 is Associated with Lipid Droplets in Mouse Liver

Global quantitative phosphoproteomics was performed by LC-MS technique in lipid droplets (LDs) isolated from the livers of high-fat diet-fed mice to identify valid constituents of the hepatic LD proteome. It has been previously shown that the LD fraction prepared by this isolation protocol is largely free of contamination as assessed by the relative enrichment of LD-resident proteins and the absence of markers that correspond to other intracellular compartments (Zhang et al. 2011). Notably Zhang failed to identify MST3 as a component of the LD fraction.

Surprisingly, the present study demonstrates that MST3, as well as phospho-MST3 (Thr¹⁷²), are present in the LD fraction, which provides the first evidence that this kinase is associated with LDs in mouse liver.

Depletion of MST3 in Human Hepatocytes Suppresses Lipid Accumulation through Increased β-Oxidation and TAG Secretion Combined with Reduced Free Fatty Acid Uptake and Lipid Synthesis

Because of its subcellular localization on liver LDs, it is concluded that MST3 regulates hepatic lipid metabolism. To investigate the possible impact of MST3 on lipid catabolism and anabolism in the liver, human IHHs were transfected with MST3-specific siRNA or with an NTC siRNA. In cells transfected with MST3 siRNA, the protein levels of MST3 were significantly reduced as assessed by Westem blot (FIG. 1, (A)).

Before metabolic assessments, the transfected cells were incubated with OA for 48 h, which is known to efficiently induce steatosis in vitro. Notably, cellular lipid storage and FFA uptake were studied both with and without OA incubation.

Knockdown of MST3 significantly reduced intrahepatocellular lipid accumulation as assessed by Oil Red O staining for neutral lipids (FIG. 1, (B)). Lipidomics analysis also confirmed significantly lower levels of several lipid species in cells transfected with MST3 siRNA compared with NTC siRNA, which was evident both under basal conditions and after challenge with OA (FIG. 2, (A)). Silencing of MST3 mediated by siRNA resulted in a marked increase in β-oxidation (FIG. 2 (B)). Furthermore, the secretion of TAG into the media was significantly higher in cells transfected with MST3 siRNA (FIG. 3, (A)). A marked reduction in FFA influx in MST3-deficient cells was also found (FIG. 3, (B)) and the incorporation of media-derived [¹⁴C]-labelled OA and [¹⁴C]-labelled glucose into intracellular TAG was lower in hepatocytes in which MST3 was depleted (FIG. 4, (A)-(B)).

### Discussion

In this study, the present inventor provides the first evidence that MST3 is associated with intracellular lipid droplets in liver. Hepatic lipid droplets, once thought to be only inert energy storage depots, are increasingly recognized as organelles that play a key role in the regulation of liver lipid partitioning (Mashek et al. 2015) providing a substrate for mitochondrial β-oxidation and secretion of very low-density lipoproteins (VLDL)-triacylglycerol (TAG). Because of its localization on liver lipid droplets, we hypothesized that MST3 regulates hepatic lipid storage. Indeed, it is demonstrated that siRNA knockdown of MST3 in human cultured hepatocytes significantly reduced intracellular lipid accumulation. There are several possible mechanisms that could underlie the suppression in lipid storage in hepatocytes where MST3 is deleted: (i) reduced rates of lipid uptake and synthesis, (ii) increased lipid secretion as TAG-rich VLDLs, (iii) enhanced levels of fatty acid oxidation—or any combination of these mechanisms. Indeed, it is demonstrated that MST3 regulates the metabolic balance of lipid catabolism versus lipid anabolism in hepatocytes, as it is demonstrated that depletion of MST3 stimulated β-oxidation and TAG secretion and inhibited FFA influx and TAG synthesis (FIG. 5).

In summary, the present disclosure provides consistent evidence for a cell-specific role of lipid droplet-associated protein MST3 in regulation of liver lipid storage. Importantly, large body of recent evidence suggests that ectopic lipid accumulation in the liver, also known as nonalcoholic fatty liver disease (NAFLD), exacerbates hepatic and systemic insulin resistance and actively contributes to the pathogenesis of type 2 diabetes and metabolic syndrome (Anstee et al. 2013).

Furthermore, hepatic lipid storage is the main risk factor for development of aggressive liver disease nonalcoholic steatohepatitis (NASH) (Anstee et al. 2013).

Consequently, the present disclosure highlights MST3 modulators as potential drug candidates for the prevention and treatment of NAFLD/NASH and related complex metabolic diseases.

### REFERENCES

Amrutkar M, Kern M, Nunez-Duran E, Stahlman M, Cansby E, Chursa U, Stenfeldt E, Boren J, Bluher M, and Mahlapuu M. 2016. Protein kinase STK25 controls lipid partitioning in hepatocytes and correlates with liver fat content in humans. Diabetologia 59, 341-353.

Anstee Q M, Targher G, and Day C P. 2013. Progression of NAFLD to diabetes mellitus, cardiovascular disease or cirrhosis. Nat. Rev. Gastroenterol. Hepatol. 10, 330-344.

Chursa U, Nunez-Duran E, Cansby E, Amrutkar M, Sutt S, Stahlman M, Olsson B M, Boren J, Johansson M E, Backhed F, Johansson B R, Sihlbom C, and Mahlapuu M. (2017) Overexpression of protein kinase STK25 in mice exacerbates ectopic lipid accumulation, mitochondrial dysfunction and insulin resistance in skeletal muscle. Diabetologia 60, 553-567.

Creasy C L, Ambrose D M, and Chernoff J. 1996. The Ste20-like protein kinase, Mst1, dimerizes and contains an inhibitory domain. J. Biol. Chem. 271:21049-21053.

Davoli T, Xu A W, Mengwasser K E, Sack L M, Yoon J C, Park P J, and Elledge S J. 2013. Cumulative haploinsufficiency and triplosensitivity drive aneuploidy patterns and shape the cancer genome. Cell. 155:948-962.

Folch J, Lees M, and Sloane Stanley G H. 1957. A simple method for the isolation and purification of total lipides from animal tissues. J. Biol. Chem. 226, 497-509.

Glatter T, Wepf A, Aebersold R, and Gstaiger M. 2009. An integrated workflow for charting the human interaction proteome: insights into the PP2A system. Mol. Syst. Biol. 5:237.

Huang C Y, Wu Y M, Hsu C Y, Lee W S, Lai M D, Lu T J, Huang C L, Leu T H, Shih H M, Fang H I, et al. 2002. Caspase activation of mammalian sterile 20-like kinase 3 (Mst3). Nuclear translocation and induction of apoptosis. J. Biol. Chem. 277:34367-34374.

Kean M J, Ceccarelli D F, Goudreault M, Sanches M, Tate S, Larsen B. Gibson L C, Derry W B, Scott I C, Pelletier L, et al. 2011. Structure-function analysis of core STRIPAK Proteins: a signaling complex implicated in Golgi polarization. J. Biol. Chem. 286:25065-25075.

Lee K K, Murakawa M, Nishida E, Tsubuki S, Kawashima S, Sakamaki K, and Yonehara S. 1998. Proteolytic activation of MST/Krs, STE20-related protein kinase, by caspase during apoptosis. Oncogene. 16:3029-3037.

Lee K K, Ohyama T, Yajima N, Tsubuki S, and Yonehara S. 2001. MST, a physiological caspase substrate, highly sensitizes apoptosis both upstream and downstream of caspase activation. J. Biol. Chem. 276:19276-19285.

Lee W S, Hsu C Y, Wang P L, Huang C Y, Chang C H, and Yuan C J. 2004. Identification and characterization of the nuclear import and export signals of the mammalian Ste20-like protein kinase 3. FEBS Lett. 572:41-45.

Lu T J, Lai W Y, Huang C Y, Hsieh W J, Yu J S, Hsieh Y J, Chang W T, Leu T H, Chang W C, Chuang W J, et al. 2006. Inhibition of cell migration by autophosphorylated mammalian sterile 20-like kinase 3 (MST3) involves paxillin and protein-tyrosine phosphatase-PEST. J. Biol. Chem. 281:38405-38417.

Madsen C D, Hooper S, Tozluoglu M, Bruckbauer A, Fletcher G, Erler J T, Bates P A, Thompson B, and Sahai E. 2015. STRIPAK components determine mode of cancer cell migration and metastasis. Nat. Cell Biol. 17:68-80.

Mashek D G, Khan S A, Sathyanarayan A, Ploeger J M, and Franklin M P. 2015. Hepatic lipid droplet biology: Getting to the root of fatty liver. Hepatology 62, 964-967.

Nerstedt A, Cansby E, Andersson C X, et al. 2012. Serine/threonine protein kinase 25 (STK25): a novel negative regulator of lipid and glucose metabolism in rodent and human skeletal muscle. Diabetologia 55:1797-1807.

Olesen S H, Zhu J Y, and Martin M P. 2016. Discovery of Diverse Small-Molecule Inhibitors of Mammalian Sterile20-like Kinase 3 (MST3). Chem. Med. Chem. 11, 1137-1144.

Stahlman M, Fagerberg B, Adiels M, Ekroos K, Chapman J M, Kontush A, and Boren J. 2013. Dyslipidemia, but not hyperglycemia and insulin resistance, is associated with marked alterations in the HDL lipidome in type 2 diabetic subjects in the DIWA cohort: impact on small HDL particles. Biochim Biophys Acta 1831, 1609-161.

Stockton R A, Shenkar R, Awad I A, and Ginsberg M H. 2010. Cerebral cavernous malformations proteins inhibit Rho kinase to stabilize vascular integrity. J. Exp. Med. 207:881-896.

Thompson B J and Sahai E. 2015. MST kinases in development and disease. J. Cell Biol. 210 (6), 871-882.

Tozluoglu M, Mao Y, Bates P A, and Sahai E. 2015. Cost-benefit analysis of the mechanisms that enable migrating cells to sustain motility upon changes in matrix environments. J. R. Soc. Interface. 12. 10.1098/rsif.2014.1355.

Ultanir S K, Yadav S, Hertz N T, Oses-Prieto J A, Claxton S, Burlingame A L, Shokat K M, Jan L Y, and Jan Y N. 2014. MST3 kinase phosphorylates TAO1/2 to enable Myosin Va function in promoting spine synapse development. Neuron. 84:968-982.

Zheng X, Xu C, Di Lorenzo A, Kleaveland B, Zou Z, Seiler C, Chen M, Cheng L, Xiao J, He J, et al. 2010. CCM3 signaling through sterile 20-like kinases plays an essential role during zebrafish cardiovascular development and cerebral cavernous malformations. J. Clin. Invest. 120:2795-2804.

Zhang H, Wang Y, Li J, Yu J, Pu J, Li L, Zhang H, Zhang S, Peng G, Yang F, and Liu P. 2011. Proteome of skeletal muscle lipid droplet reveals association with mitochondria and apolipoprotein a-I. J. Proteome. Res 10, 4757-4768.

### REFERENCE TO A “SEQUENCE LISTING” SUBMITTED AS AN XML FILE

The material in the XML file, named “NOVI-71218US2-Sequence-Listing.xml”, created Feb. 26, 2025, file size of 8,192 bytes, is hereby incorporated by reference.

1. A method for treating or preventing a metabolic disease in a subject, the method comprising administering a pharmaceutically effective amount of an oligonucleotide or polynucleotide 8-80 bases in length, wherein the oligonucleotide or polynucleotide is hybridizable to the nucleic acid sequence of SEQ ID NO: 1, or a sequence complementary to SEQ ID NO:1, thereby suppressing intracellular accumulation of cholesteryl ester and treating the subject having a metabolic disease, and wherein the metabolic disease is selected from the group consisting of non-alcoholic fatty liver disease (NAFLD), non-alcoholic steatohepatitis (NASH), fatty pancreas disease, and fatty kidney disease.

2. The method of claim 1, wherein the metabolic disease is non-alcoholic fatty liver disease (NAFLD).

3. The method of claim 1, wherein the metabolic disease is non-alcoholic steatohepatitis (NASH).

4. The method of claim 1, wherein the oligonucleotide or polynucleotide is selected from the group consisting of siRNA, shRNA, and antisense RNA or DNA.

5. The method of claim 4, wherein the oligonucleotide or polynucleotide is antisense RNA or DNA.

6. The method of claim 5, wherein the antisense oligonucleotide or polynucleotide is 8-30 bases in length.

7. The method of claim 6, wherein the subject is human.

8. The method of claim 1, wherein a nucleotide in the oligonucleotide or polynucleotide comprises an RNA residue, a DNA residue, or a nucleotide analogue.

9. The method of claim 1, wherein the oligonucleotide or polynucleotide is selected from the group consisting of a peptide nucleic acid (PNA), a phosphorodiamidate morpholino oligomer (PMO), and a phosphorothioate oligonucleotide (PS-ODN).
